(19) United States
(12) Patent Application Publication
(10) Pub. No.: US 2014/0322144 A1
(43) Pub. Date: Oct. 30, 2014

(54) ALKALINE COMPOSITIONS AND THEIR DENTAL AND MEDICAL USE

(71) Applicants: Basil Athanassiadis, Annerley (AU); Laurence James Walsh, Ferny Hills (AU)

(72) Inventors: Basil Athanassiadis, Annerley (AU); Laurence James Walsh, Ferny Hills (AU)

(73) Assignees: LJ WALSH DENTAL PTY LTD, Ferny Hills, Queensland (AU); B ATHANASSIADIS DENTAL PTY LTD, Annerley, Queensland (AU)

(21) Appl. No.: 14/235,160

(22) PCT Filed: Jul. 27, 2012
(86) PCT No.: PCT/AU2012/000895

§ 371 Date: Jun. 3, 2014

(30) Foreign Application Priority Data
Jul. 27, 2011 (AU) 2011902984

(51) Int. Cl. A61K 6/00; A61Q 11/00; A61K 8/19; A61K 31/05; A61K 31/192

(52) U.S. Cl. CPC A61K 6/0067; A61K 31/05; A61K 31/192; A61K 8/19; A61Q 11/00; A61K 6/0038; A61K 6/0035
USPC 424/49; 424/689

(57) ABSTRACT

The present invention relates to antimicrobial alkaline compositions suitable for use in medical and dental treatment. The compositions comprise a polyalkylene glycol or C₃-C₆ polyol compound and calcium hydroxide or components that generate calcium hydroxide in situ. The use of the compositions as dental medicaments, obturants, liners, oral disinfectants and bone cements is also described.

### FIELD OF THE INVENTION

The present invention relates generally to alkaline compositions that have antimicrobial activity, including compositions that comprise calcium hydroxide and either a polyalkylene glycol or a C₃-C₆ diol or triol compound, and methods of using the compositions in dental treatment both human and veterinary, and as bone cements for medicinal and veterinary use. The present invention also relates to compositions comprising components that generate calcium hydroxide in situ which then reacts with the C₃-C₆ polyol compound. The present invention also relates to capsules and kits comprising the components of the compositions, and to methods of making the compositions.

### BACKGROUND OF THE INVENTION

The reference in this specification to any prior publication (or information derived from it), or to any matter which is known, is not, and should not be taken as an acknowledgment or admission or any form of suggestion that that prior publication (or information derived from it) or known matter forms part of the common general knowledge in, the field of endeavour to which this specification relates.

Endodontic treatment in both humans and animals, seeks to avoid extracting damaged, infected or diseased teeth. Normally dental pulp is sterile, and if this tissue becomes infected or necrotic it can become a potential reservoir for bacteria. Since bacteria play a primary role in the initiation and progression of endodontic diseases, the presence of bacteria within the root canal system is linked fundamentally to poor clinical outcomes. Therefore, endodontic treatment aims to eradicate the bacteria inside teeth, seal the root canals and prevent recontamination of dental tissues.

One of the first steps in endodontic treatment is disinfection of the root canal by mechanical debridement of the canal wall and subsequent application of an antiseptic substance within the canal to kill the remaining bacteria. However, it is not possible to create a sterile space in all teeth with infected root canals due to the complexity of root canal systems, and the inability of instruments to contact all surfaces of the root canals (Byström and Sundqvist, Scandinavian Journal of Dental Research 1981; 89:321-8; Byström et al Endodontic and Dental Traumatology 1985; 1:70-5).

In particular, the root canal includes irregular spaces such as isthmuses, ramifications, accessory canals and apical deltas. Furthermore, the wall surrounding a root canal consists of dentine, a calcified organic matrix through which approximately 30,000 tubules/square mm pass horizontally from the root canal space towards the outer surface of the root. These tubules typically have a diameter of 2-5 micrometres, are not uniform in shape or size throughout the length of the root, and are also filled with organic matter. Bacteria which have a diameter of 1 micron easily penetrate, survive and even proliferate in these tubular structures as well as in the root canal and irregular spaces.

This is further complicated because root canal infections are often polymicrobial. Typically these infections include one or more bacteria selected from Peptostreptococcus spp., Prevotella spp., Porphyromonas spp., Fusobacterium spp., Eubacterium spp., Actinomyces spp., Bacteroides spp. and facultative Streptococcus spp. In cases of infected canals from previously root filled teeth the most common bacteria found include Enterococcus spp., Streptococcus spp. and Lactobacillus spp. The bacteria may be found not only in planktonic forms, but also as adherent well-organised biofilms which are more resistant to antimicrobial agents.

After mechanical debridement and application of an antiseptic, the use of intracanal medicaments is therefore recommended to prevent repopulation of the root canals with residual bacteria. It is also recommended to leave the medicament in the root canal for substantial periods of time, which means that the treatment of infected root canals is completed in more than one visit (Byström et al Endodontics and Dental Traumatology 1985; 1:70-5; Chong and Pitt Ford, International Endodontic Journal 1992; 25:97-106).

Intracanal medicaments should therefore include an antimicrobial agent which is broad spectrum covering Gram-negative bacteria, Gram-positive bacteria and fungi. Furthermore, the medicament needs to be easily removed, as after a period of treatment the medicament is replaced with an obturant to permanently fill the root canal. None of the current commercial products meet these requirements.

Obturants are used to permanently fill the root canal after treatment with a medicament. Obturants must be resorbable if they are to be used with deciduous teeth. Furthermore obturants need to set hard enough to provide a stable situation in the root canal, but not be difficult to work with and to remove, if required. Many commercially available obturants do not meet these requirements. For example, mineral trioxide aggregate (MTA) is not resorbable and therefore cannot be used with deciduous teeth as an endodontic obturant within the root canals. Zinc oxide eugenol (ZOE) is one of the most commonly used obturants in deciduous teeth, but ZOE has limited antimicrobial action, has a slow rate of resorption, has a tendency to be retained even after tooth exfoliation, and in some cases unresorbed material has been found to cause deflection of the succedaneous permanent tooth. Iodoform pastes, such as Kripaste, may also be used as obturants in deciduous teeth. However, Kripaste can be difficult to remove as it does not set hard, and iodoform pastes may produce a yellowish-brown discoloration of tooth crowns, which may compromise aesthetics.

There is a need to provide medicament compositions which have broad spectrum antibacterial activity and are easily removed after use as well as obturant compositions that are hard setting but able to be worked with and removed, have antimicrobial properties, if necessary, are resorbable and do not compromise aesthetics.

Similar considerations with regard to the need for broad spectrum antibacterial activity arise with regard to dental materials used to line deep cavities where they may be microscopic or macroscopic exposures of the dental pulp tissues. Because the lactic acid-affected dentine underlying a deep cavity is a low pH environment where acid-tolerant bacteria flourish, an ideal lining material for deep cavities in teeth would have high release of hydroxyl ions and would create an alkaline pH which would be highly unfavourable for the continued viability of acid-tolerant bacteria.

Current materials used to line deep cavities or which are placed on exposed dental pulp tissue are based on calcium hydroxide in water-based vehicles, which is problematic because the low solubility of calcium hydroxide in water limits the release of hydroxyl ions. MTA and related materials such as Biodentine (Septodont, Paris) which are based on tricalcium silicate and calcium oxide have been introduced recently, as derivatives from the chemistry of industrial Portland cement. The major limitations of these materials are their complex and expensive manufacturing processes, their long setting times, their awkward handling properties during manipulation by the dentist, and stringent requirements in terms of the ratio of water to powder. Biodentine cannot be mixed by hand, while MTA has the additional problem of causing discolouration of tooth structure.

Because of such issues, there is a need for materials which have alkaline properties, are suitable for use in deep cavities, are easy to manipulate by the dentist, and do not cause staining of teeth.

Bone cements are used to anchor artificial joints, such as hip, knee, shoulder and elbow joints, in place. A risk with any type of surgery, is infection at the surgical site with bacteria or fungi. Therefore there is a need for bone cements with antimicrobial properties that reduce the risk of infection.

### SUMMARY OF THE INVENTION

The present invention is predicated in part on the discovery that calcium hydroxide, either present in the composition or generated in situ, can be combined with solvents such as glycerol, propylene glycol and polyethylene glycol to provide liquid or paste compositions that contain higher amounts of calcium hydroxide than can be contained in aqueous compositions and thereby maintain greater hydroxyl ion release and higher pH in preparation and in use. Medicament compositions comprising calcium hydroxide and solvents such as polyethylene glycol or polypropylene glycol have a pH of at least 13 and have broad spectrum antimicrobial activity and desirable handling and storage properties because dehydration problems are eliminated. Obturant compositions formed from a calcium hydroxide source and a solvent such as glycerol or propylene glycol in approximately 1:1 ratio sets hard and has suitable properties for use as an obturant that has antimicrobial properties.

Similarly, chemical reactions which generate calcium hydroxide can be used not only for manufacturing suitable preparations for use as components in the compositions of the invention, but can directly contribute to the formation of suitable materials for root canal obturants and liners of deep cavities. Calcium hydroxide nanoparticles generated by chemical reactions can also be used in aqueous vehicles with co-solvents, allowing greater hydroxyl ion release, with use for rinsing root canals or as a dental mouthrinse. Appropriate selection of flavouring agents makes such compositions palatable and suitable for use even in individuals with low tolerance for conventional mouthrinses.

The present invention is also predicated in part on the discovery that a source of calcium hydroxide can be included in dental resins and bone cements to impart antimicrobial properties.

### DESCRIPTION OF THE INVENTION

In one aspect, the present invention provides a composition comprising a polyalkylene glycol solvent such as a polyethylene glycol or polypropylene glycol solvent and calcium hydroxide, wherein the pH of the composition is at least 13.0. This composition is suitable for use as an endodontic medicament. In the description below this composition is referred to as “the medicament composition”.

The medicament composition comprises calcium hydroxide in suitable solvent which is optionally lacking in water (water-free). Calcium hydroxide is bactericidal, is antifungal, can penetrate biofilms, can stimulate the repair of dentine and can withstand the buffering effect of dentine. In contrast, many endodontic medicaments comprise antibiotic/steroid combinations. Medicaments comprising antibiotic/steroid combinations are typically bacteriostatic, are not antifungal, have a limited ability to penetrate biofilms, are not involved in dentine repair, and their spectrum of activity is limited by the buffering effect of dentine. Furthermore, calcium hydroxide has the ability to kill a broader range of bacteria than antibiotic/steroid combinations. Bacteria have been reported to develop resistance to antibiotic/steroid combinations, in contrast to calcium hydroxide where resistance has not been reported.

While in some embodiments the calcium hydroxide may be used in granular or powder form having particulate size, in the micron range or aggregates of such particles, in other embodiments, the calcium hydroxide may be in nanoparticulate form, having an average particulate size between 20 nanometers and 500 nanometers. Advantageously, the use of nanoparticulate calcium hydroxide increases the surface area of the calcium hydroxide and therefore maintains a highly alkaline pH even under dilution conditions, and improves dissolution of the calcium hydroxide into the polyalkylene glycol.

The nanoparticulate calcium hydroxide can be prepared by methods known in the art. For example, small quantities may be prepared by heating solutions of calcium chloride and sodium hydroxide to between 40° C. to 90° C. and then slowly adding the sodium hydroxide solution, for example, dropwise, to the warm calcium chloride solution. Another method is described by Danielea and Taglieri, Journal of Cultural Heritage, 2011, doi:10.1016/j.culher.20111.05.007). This method allows the bulk addition of hot calcium chloride solution to hot sodium hydroxide solution by addition of a non-ionic or anionic surfactant to the calcium chloride solution. The non-ionic surfactant may be any surfactant from the Tween, Triton or Brij series, ethoxylates based on polyoxyethylene or glycosides such as thioglycosides or maltosides (HEGA and MEGA series surfactants). Suitable anionic surfactants include sodium dodecyl sulfate, ammonium lauryl sulfate and sodium dodecyl benzene sulfonate, especially sodium dodecyl sulfate.

The surfactant present is present in the calcium chloride solution in an amount of from 0.1 to 2.0% w/w of the calcium chloride solution, especially about 1.0% w/w.

The nanoparticulate calcium hydroxide may be isolated by decanting the supernatant from the particulate or by filtration. The calcium hydroxide nanoparticulate material may also be washed with water to remove excess calcium chloride, sodium hydroxide and surfactant.

In some embodiments, the medicament composition comprises from 25% to 55% calcium hydroxide, more especially from 30% to 50% calcium hydroxide, most especially from 35% to 45% calcium hydroxide.

While calcium hydroxide has previously been used in endodontic medicaments, such Medicaments typically have a pH of below 12.6. For example, calcium hydroxide pastes (20-50%) in water with barium sulfate and methyl cellulose or carboxymethylcellulose have a pH in the range of 11.8 to 12.6. The dentine in the root canal buffers this type of medicament lowering the pH after application to about 7-8. While not wishing to be bound by theory, it is thought that the presence of a polyalkylene glycol solvent in the medicament composition results in a higher pH from a greater concentration of available hydroxide ions, hence a higher pH will be achieved once the medicament composition is buffered by the dentine and this will maintain the antimicrobial properties of calcium hydroxide.

The pH of the medicament composition is at least 13.0, especially at least 13.2 or 13.5, more especially at least 13.7, and most especially at least 14.0. Without wishing to be bound by theory, it is believed that calcium hydroxide is more soluble in polyalkylene glycols than in water and therefore a greater pH can be achieved. Furthermore, the 0-14 pH range is relevant to acidic and basic aqueous solutions at room temperature. pH values greater than 14 can be achieved in non-aqueous compositions for example, pH values of 14.11 have been observed with compositions of calcium hydroxide in polyethylene glycol.

Advantageously, calcium hydroxide may be formulated into a medicament composition using a polyalkylene glycol solvent. The medicament composition of the present invention comprising a polyalkylene glycol solvent typically does not dry out when stored, and may be easily handled prior to and during administration. Many known endodontic medicaments use methylcellulose as a base, which tends to dry out over time. Furthermore, endodontic medicaments comprising methylcellulose may not be stable for extended periods of time.

The polyalkylene glycol solvent in the medicament composition is any polyalkylene glycol solvent which is in liquid form. The polyalkylene glycols are those recognised as safe for use in medical or food applications. In particular embodiments, the polyalkylene glycol is selected from polyC₂-C₃alkylene glycols, for example, polyethylene glycol (PEG) and polypropylene glycol (PPG). Polyalkylene glycols are polymeric ethers and therefore come in a variety of different molecular weights. Polyalkylene glycols may have a number that approximately corresponds to their molecular weight. In some embodiments, the polyalkylene glycol solvent has a molecular weight of 700 or less or a mixture thereof; especially a polyalkylene glycol with a molecular weight of from 100 to 700 or a mixture thereof; or a polyalkylene glycol with a molecular weight of from 200 to 600 or a mixture thereof; more especially a polyalkylene glycol with a molecular weight of from 300 to 500 or a mixture thereof; most especially polyalkylene glycol 400. The polyalkylene glycol solvent may be selected from PEG 200, PEG 300, PEG 400, PEG 500, PEG 600, PEG 700, PPG 200, PPG 300, PPG 400, PPG 500, PPG 600 and PPG 700 or a combination thereof; especially from PEG 200, PEG 300, PEG 400, PEG 500, PEG 600, PPG 200, PPG 300, PPG 400, PPG 500 and PPG 600 or a combination thereof; more especially from PEG 300, PEG 400, PEG 500, PPG 300, PPG 400 and PPG 500 or a combination thereof; most especially PEG 400 or PPG 400.

In some embodiments, the medicament composition comprises from 30 to 60% of the polyalkylene glycol solvent; especially from 35% to 55%; more especially from 40% to 50%; or from 42-48%; most especially about 45% of polyalkylene glycol solvent.

In some embodiments, the medicament composition further comprises a viscosity modifier. The viscosity modifier may be a polyalkylene glycol polymer with a molecular weight of at least 1000, especially a polyethylene glycol or polypropylene glycol with a molecular weight of at least 1000. In some embodiments, the viscosity modifier is a PEG or PPG with a molecular weight of from 2300 to 6000 or a mixture thereof; especially a PEG or PPG with a molecular weight of from 2600 to 4000 or a mixture thereof; or a PEG or PPG with a molecular weight of from 2800 to 4000 or a mixture thereof; or a PEG or PPG with a molecular weight of from 3000 to 3750 or a mixture thereof; more especially a PEG or PPG with a molecular weight of from 3250 to 3500 or a mixture thereof; most especially PEG 3350 or PPG 3350. Exemplary polyalkylene glycol viscosity modifiers include PEG 2300, PEG 2400, PEG 2500, PEG 2600, PEG 2700, PEG 2800, PEG 2900, PEG 3000, PEG 3250, PEG 3350, PEG 3500, PEG 3750, PEG 4000, PPG 1000, PPG 1200, PPG 2000, PPG 3000 and PPG 4000.

Use of higher molecular weight viscosity modifiers, especially PEG, provides medicament compositions with higher viscosities and these compositions may become firm if maintained for several hours at 2-4° C. Such medicament compositions may be suitable for use in pastilles.

While not wishing to be bound by theory, it is believed that the viscosity of the medicament composition affects the rate of ionic dissociation of calcium hydroxide. Typically the lower the viscosity of the composition, the higher the rate of ionic dissociation of calcium hydroxide and the greater the hydroxyl ion release. Higher viscosity compositions may minimise the physical dispersion of calcium hydroxide medicament into the dental tissues and maintain the paste in the desired area for longer periods of time.

In some embodiments, the medicament composition comprises from 1% to 30% of a viscosity modifier. If a low viscosity product is desired, the medicament composition may comprise from 1% to 10%; especially from 2% to 7.5%; more especially from 3% to 5%; most especially about 4% viscosity modifier.

For a medium viscosity composition, the medicament composition may comprise from 5 to 20% viscosity modifier; more especially from 7 to 18% viscosity modifier; or from 10 to 15% viscosity modifier; most especially about 12.5% viscosity modifier.

If a high viscosity composition is desired, the medicament composition may comprise from 15% to 30% viscosity modifier; especially from 17% to 28%; more especially from 20% to 25%; most especially about 22.5% viscosity modifier.

The viscosity may also be modified by the molecular weight of the viscosity modifier. For example, a high molecular weight viscosity modifier such as PEG 6000 will increase viscosity to a greater extent than a lower molecular weight viscosity modifier such as PEG 2300. The desired viscosity may be obtained by selecting a specific amount of a specific molecular weight viscosity modifier. Variation in viscosity may be achieved by varying the amount and/or molecular weight of the viscosity modifier.

The medicament composition may also further comprise a radiopaquing agent. Exemplary radiopaquing agents include strontium, zirconium, lanthanum, tungsten, bismuth or barium compounds; especially zirconium or barium compounds; more especially zirconium compounds. In some embodiments, the radiopaquing agent is selected from strontium oxide, zirconium silicate, zirconium oxide, zirconium dioxide, lanthanum oxide, calcium tungstate, bismuth oxide, barium zirconate and barium sulphate or a combination thereof; more especially zirconium dioxide and barium sulphate; most especially zirconium dioxide.

In some embodiments, the medicament composition comprises from 10% to 20% radiopaquing agent, especially from 12% to 18% radiopaquing agent; more especially about 15% radiopaquing agent.

The medicament composition may also further comprise an essential oil. Suitable essential oils include terpenes and terpenoids, for example, terpen-4-ol, cymene, sabinene, alpha pinene, beta pinene, citronellol, geraniol, carvacrol, thymol, farnesol and caryophyllene, and aromatic and aliphatic essential oils, for example, cinnamaldehyde, cinnamyl alcohol, chavicol, eugenol, amethole, estragole, safrole, ascaridole and menthol. In some embodiments, the composition comprises a mixture of essential oils. In particular embodiments, the essential oil has both antimicrobial and/or anti-inflammatory properties. In particular embodiments, the essential oil comprises terpinen-4-ol, cinnamaldehyde or carvacrol, or a mixture thereof. In one embodiment, the medicament composition may comprise up to 25% essential oil, especially up to 20% essential oil, or up to 15% essential oil, or up to 10% essential oil. For example, the medicament composition may comprise from 2% to 25% essential oil, or from 5% to 20% essential oil, or from 10% to 15% essential oil. In some embodiments, the medicament composition comprises from 2% to 25%, especially 10% to 15% terpinen-4-ol, or up to 10% carvacrol or cinnamaldehyde. In some embodiments, the essential oil is solubilised in the composition by the addition of a surfactant, especially a non-ionic surfactant that can solubilise oils in hydrophilic substances. One particularly useful surfactant is Mysol™381.

In one embodiment, the medicament composition further comprises an anti-inflammatory agent. In some embodiments, the anti-inflammatory agent is a nonsteroidal anti-inflammatory drug (NSAID) or a corticosteroid, especially a NSAID. Exemplary NSAIDs include ibuprofen, dexibuprofen and lysine ibuprofen, especially ibuprofen and dexibuprofen, more especially dexibuprofen.

The medicament composition may also further comprise a colourant. The colourant may produce a medicament with a white colour, and may be especially a titanium compound, for example, titanium dioxide. In some embodiments, the medicament composition comprises between 0.5% and 5% colourant, especially about 2% colourant.

In some embodiments, the medicament compositions may have a neutral, non-penetrating colour, especially if the composition comprises, for example, a colourant that is titanium dioxide, and a radiopaquing agent that is zirconium dioxide or barium sulphate.

In some embodiments, the medicament composition further comprises one or more vehicles. Exemplary vehicles are selected from glycerol; propylene glycol, acids such as oleic acid; water; ethanol; silicone-based materials (both non-volatile and volatile) such as cyclomethicone, dimethicone and dimethiconecopolyol; hydrocarbons such as squalane, squalene and petrolatum, especially squalane and petrolatum; a sustained release vehicle such as a microsponge or a polymer matrix; surfactants, such as cationic and amphoteric surfactants; a stabilising agent; a suspending agent; an emulsifying agent; a pH regulator such as a citrate or a phosphate salt; or a combination thereof. Other vehicles suitable for use with the compositions would also be known from the cosmetic and pharmaceutical arts.

Mixtures of water and organic solvents (such as water and ethanol), and mixtures of ethanol and glycerol or propylene glycol may also be present in the medicament composition. In some embodiments, the medicament composition comprises up to 5% water, especially up to 3% water, more especially up to 1% water, and most especially the medicament composition does not comprise water.

In another aspect, the present invention relates to a method of making the medicament composition described above. This method comprises adding calcium hydroxide to a polyalkylene glycol solvent, and mixing the combination. This combination should be mixed for a period of at least 5 minutes to provide a homogenous composition. In other embodiments, this method further comprises mixing one or more of a viscosity modifier, a radiopaquing agent, an essential oil, an anti-inflammatory agent, a colourant and a vehicle into the polyalkylene glycol solvent or into the calcium hydroxide-polyalkylene glycol solvent combination.

In one example, to provide a medium viscosity paste deionized water is optionally added to PEG 400 in a stainless steel vessel. Water typically comprises 0-5% of the total volume. PEG 3350 is then added, and the mixture is heated (for example to 50-70° C.) with constant stirring until the PEG 3350 melts and dissolves evenly into the mixture. The mixture is then removed from heating and the following are added (with gentle stirring), especially in sequence: calcium hydroxide powder, zirconium dioxide powder, and titanium dioxide powder. The mixture is then agitated mechanically for 5 minutes, and is then dispensed into tubes or syringes as appropriate.

In another aspect, the present invention provides the medicament composition described above for use in the treatment or prevention of a microbial infection in a tooth, painful extirpation, “hot pulp” syndrome or acute pulpitis, or a combination thereof; especially in the treatment or prevention of a microbial infection in a tooth.

In a further aspect, the present invention provides a method of treating or preventing a microbial infection in a tooth, painful extirpation, “hot pulp” syndrome or acute pulpitis, or a combination thereof (especially a microbial infection in a tooth), comprising administering the medicament composition described above to a subject. The medicament composition is especially administered into the root canal of the subject.

In another aspect, the present invention provides a use of calcium hydroxide and a polyethylene glycol solvent in the manufacture of the medicament composition described above for the prevention or treatment of a microbial infection in a tooth, painful extirpation, “hot pulp” syndrome or acute pulpitis, or a combination thereof (especially a microbial infection in a tooth), wherein the medicament has a pH of at least 13.0.

In particular embodiments, the microbial infection is caused by Enterococcus spp., Streptococcus spp., Lactobacillus spp., Peptostreptococcus spp., Prevotella spp., Porphyromonas spp., Fusobacterium spp., Eubacterium spp., Actinomyces spp., Bacteroides spp., and Candida spp., especially Enterococcus spp. such as E. Faecalis and Candida spp. such as Candida albicans.

In another aspect, the present invention provides a composition comprising a C₃-C₆ polyol and a powder component, wherein the powder component comprises calcium hydroxide, the C₃-C₆ polyol being present in an amount of from 35% to 65% of the composition, and wherein the powder component being present in an amount of from 65% to 35% of the composition. This composition is suitable for use as an endodontic obturant and/or liner for cavity preparation, and in the description below this composition is referred to as “the obturant composition”.

Advantageously, it has been found that a composition comprising 45% to 55% C₃-C₆ polyol and 45% to 55% of a powder component (which comprises or generates calcium hydroxide), sets to form a hard composition and is suitable for use as an obturant composition. The setting reaction occurs at a rate suitable for the clinical manipulation of the material by the dental practitioner, and results in a set product which is rigid and which provides a seal against the walls of the root canal, resisting the passage of oral fluids should the tooth later experience leakage from fracture, recurrent decay or other causes. In various aspects, the obturant composition is advantageously hard enough to provide a stable situation in the root canal, but is easy to work with. For example, the obturant composition once set in the root canal may be drilled away or chipped with an ultrasonic scaler or drill, and it may be possible to vibrate or drill the obturant composition out of the tooth after the composition has set. Furthermore, the obturant composition may be resorbable, permitting its use with deciduous teeth and in veterinary dentistry. The obturant composition also may be relatively inexpensive to manufacture. A particularly useful feature is the high release of hydroxide ions by the set obturant material when in contact with water, providing an antibacterial action from the high pH created.

In some embodiments, a lower amount of C₃-C₆ polyol may be used to provide a composition suitable for use in lining cavities before filling. For example compositions having 35% to 45% C₃-C₆ polyol and 65% to 55% powder are suitable liner compositions.

As used herein “C₃-C₆ polyol” refers to a compound having 3 to 6 carbon atoms and at least two hydroxyl groups. In particular embodiments, the polyol is a diol having two hydroxy groups or a triol having three hydroxy groups. In particular embodiments the polyol is a C₃ polyol. Examples of suitable polyols include glycerol and propylene glycol.

The amount of C₃-C₆ polyol in the obturant composition is from 45% to 55% of the composition, especially from 45% to 53% of the composition, more especially from 47 to 53% of the composition, or from 45% to 50% of the composition, most especially about 50% of the composition.

The amount of C₃-C₆ polyol in the liner composition is ideally from 35% to 45% of the composition, especially 35% to 40%, more especially 37% to 38% of the composition.

The term “powder component” refers to the proportion of the composition which comprises one or more solid or semi solid ingredients, especially ingredients in powder form.

The amount of the powder component in the obturant composition is from 35% to 65% of the composition, especially from 55% to 65% or 45% to 55% of the composition, more especially from 60% to 65% or 47% to 53% of the composition, or from 50% to 55% of the composition, most especially 62% to 63% or about 50% of the composition.

The powder component comprises calcium hydroxide. Advantageously, calcium hydroxide is bactericidal, is antifungal, can penetrate biofilms, can be involved in dentine repair and can withstand the buffering effect of dentine. Therefore, the obturant composition also may be effective in preventing or treating bacterial infections in a tooth.

In one embodiment, the powder component comprises from 20% to 100% calcium hydroxide, especially from 30% to 100% calcium hydroxide.

In some embodiments, the powder component consists of calcium hydroxide. In other embodiments, the powder component comprises from 20% to 60% calcium hydroxide, especially from 30% to 50% calcium hydroxide, especially about 40% calcium hydroxide.

In some embodiments, the calcium hydroxide is nanoparticulate calcium hydroxide.

In some embodiments, the powder component further comprises a iadiopaquing agent. Exemplary radiopaquing agents include strontium, zirconium, lanthanum, tungsten, bismuth or barium compounds; especially zirconium or barium compounds; more especially barium compounds. In some embodiments, the radiopaquing agent is selected from strontium oxide, zirconium silicate, zirconium oxide, zirconium dioxide, lanthanum oxide, calcium tungstate, bismuth oxide, barium zirconate and barium sulphate or a combination thereof; more especially zirconium dioxide and barium sulphate; most especially barium sulphate.

In some embodiments, the powder component comprises from 20% to 70% radiopaquing agent, especially from 30% to 60% radiopaquing agent; more especially from 45% to 55% radiopaquing agent; most especially about 50% radiopaquing agent.

The obturant composition may take about 12 hours to set hard. However, the setting time can be reduced through the use of an accelerant. Therefore, in another embodiment the powder component further comprises an accelerant. Exemplary accelerants are selected from calcium sulphate and alkali metal halogen salts; especially from calcium sulphate, synthetic anhydrite, sodium chloride and potassium chloride; more especially from calcium sulphate and sodium chloride. In particular embodiments, the sodium chloride accelerant is present in the amount of up to 5% by weight of the powder component or the calcium suphate accelerant is present in an amount of up to 17% by weight of the powder component. If calcium sulphate is used in the obturant composition as an accelerant, it is especially in the form of calcium sulphate hemihydrate before the composition sets, and in the form of calcium sulphate dihydrate after the composition sets.

Other accelerants include up to 5% by weight calcium chloride in the powder, up to 5% by weight calcium nitrite in the powder, up to 11% by weight potassium sulphate in the powder, up to 26% by weight strontium chloride, replacing the radiopaque agent, up to 5% by weight potassium thiocyanate in the powder, up to 3% by weight sodium carbonate in the powder, up to 25% by weight sodium aluminate in the powder, Portland cement, calcium-sulpho-aluminate (e.g. Calumex CSA®), calcium aluminate cement, amorphous calcium aluminate (e.g. Calumex SCA®), magnesium chloride and lithium compounds selected from lithium carbonate, lithium nitrate, lithium chloride and lithium hydroxide or a mixture of any of these accelerants.

Advantageously, the presence of an accelerant or mixtures thereof provide short setting times that are desirable for clinical practice. In some embodiments, the obturant has sufficient strength within the time of a dental appointment to be overlaid with other materials. In this specification, the composition comprising at least calcium hydroxide and an accelerant in the powder component is referred to as the “accelerated obturant composition”.

In some embodiments, the accelerant includes a cross-linking agent, a gelling agent or a desiccant; especially silica; more especially fumed (pyrogenic) silica. Fumed (pyrogenic) silica has a low bulk density but a high surface area, with viscosity increasing, thixotropic behaviour when added into the composition. Fumed (pyrogenic) silica also acts as a desiccant. The inclusion of a desiccant in the obturant composition may be especially advantageous when, for example, calcium sulphate hemihydrate is used. For example, fumed (pyrogenic) may prevent or decrease the interaction of water with calcium sulphate hemihydrate. This provides an anti-caking action which improves the flow of the powder during mixing with the polyol liquid component.

In some embodiments, the powder component comprises from 5% to 25% accelerant, especially from 7% to 20% accelerant, more especially from 10% to 15% accelerant, most especially about 10% accelerant.

In some embodiments, the obturant composition further comprises an essential oil. Suitable essential oils include terpenes and terpenoids, for example, terpen-4-ol, cymene, sabinene, alpha pinene, beta pinene, citronellol, geraniol, carvacrol, thymol, farnesol and caryophyllene, and aromatic and aliphatic essential oils, for example, cinnamaldehyde, cinnamyl alcohol, chavicol, eugenol, amethole, estragole, safrole, ascaridole and menthol. In some embodiments, the composition comprises a mixture of essential oils. In particular embodiments, the essential oil has both antimicrobial and/or anti-inflammatory properties. In particular embodiments, the essential oil comprises terpinen-4-ol, cinnamaldehyde or carvacrol, or a mixture thereof. In some embodiments, the obturant composition comprises from 0.5% to 15% essential oil, especially up to 12% essential oil, more especially up to 10% essential oil. In some embodiments, the essential oil is solubilised in the composition by the addition of surfactant, especially non-ionic surfactant. One particularly useful surfactant is Mysol™-381.

In one embodiment, the obturant composition further comprises an anti-inflammatory agent. In some embodiments, the anti-inflammatory agent is a nonsteroidal anti-inflammatory drug (NSAID) or a corticosteroid, especially a NSAID. Exemplary NSAIDs include ibuprofen, dexibuprofen and lysine ibuprofen, especially ibuprofen and dexibuprofen, especially dexibuprofen.

In a further aspect, the present invention provides a capsule comprising C₃-C₆ polyol and the powder component defined above, wherein C₃-C₆ polyol is in an amount of from 35% to 65% of the material in the capsule and wherein the powder component is in an amount of from 65% to 35% of the material in the capsule. In the capsule the C₃-C₆ polyol and the powder component are stored separately in different compartments. The materials may, for example, be separated by a membrane which is ruptured when the capsule is activated at the start of the mixing process. The materials in the capsule may then be mixed using an amalgamator. Exemplary capsules are similar to those used for the Fuji glass ionomer dental products made by GC Corporation (Tokyo) or the Riva dental products made by SDI (Melbourne), which use a separating membrane to prevent the liquid and powder components from reacting until the material is ready to use by the dental practitioner.

In a further aspect, the present invention provides a kit comprising C₃-C₆ polyol and the powder component defined above. In the kit the C₃-C₆ polyol and the powder component are stored separately and the C₃-C₆ polyol and the powder component may together form one or two packages. When the obturant composition is to be used, the glycerol and the powder component in the kit are combined. C₃-C₆ polyol may comprise from 35% to 65% of the material in the kit, and the powder component may comprise from 65% to 35% of the material in the kit.

In some embodiments, C₃-C₆ polyol is from 35% to 45% of the material in the capsule or 45% to 53% of the material in the capsule or the kit, especially from 47% to 53% of the material in the capsule or the kit, more especially from 35% to 40% or 45% to 50% of the material in the capsule or the kit, most especially 37% to 38% or about 50% of the material in the capsule or the kit.

Furthermore, in some embodiments, the powder component is from 55% to 65% or 47% to 55% of the material in the capsule or the kit, especially from 60% to 65% or 47% to 53% of the material in the capsule or the kit, more especially from 62% to 63% or 50% to 55% of the material in the capsule or the kit, most especially about 50% of the material in the capsule or the kit.

In other embodiments, the capsule or kit comprises an admixture of C₃-C₆ polyol and an essential oil, such as those described above, especially an essential oil with antimicrobial and/or anti-inflammatory properties. Exemplary essential oils comprise terpinen-4-ol, cinnamaldehyde or carvacrol, or a mixture thereof. In some embodiments, the essential oil comprises up to 0.5% to 15% of the material in the capsule or the kit, especially up to 10% of the material in the capsule or the kit. In some embodiments, the polyol further comprises a surfactant to assist with solubilising the essential oil, especially a non-ionic surfactant, for example, Mysol™381.

In some embodiments, the capsule or kit comprises an admixture of C₃-C₆ polyol and an anti-inflammatory agent, or the powder component in the capsule or kit further comprises an anti-inflammatory agent. In some embodiments, the anti-inflammatory agent is a nonsteroidal anti-inflammatory drug (NSAID) or a corticosteroid, especially a NSAID. Exemplary NSAIDs include ibuprofen, dexibuprofen and lysine ibuprofen, especially ibuprofen and dexibuprofen, more especially dexibuprofen.

In another aspect, the present invention provides a powder composition comprising calcium hydroxide optionally nanoparticulate calcium hydroxide and a radiopaquing agent, wherein the calcium hydroxide is in an amount of from 20% to 60% of the composition and the radiopaquing agent is in an amount of from 30% to 70% of the composition.

The powder composition comprises from 20% to 60% calcium hydroxide, especially from 30% to 50% calcium hydroxide, more especially about 40% calcium hydroxide.

Suitable radiopaquing agents are discussed above in relation to the powder component of the obturant composition. The powder composition comprises from 30% to 70% radiopaquing agent, especially from 40% to 60% radiopaquing agent; more especially from 45% to 55% radiopaquing agent; most especially about 50% radiopaquing agent. The radiopaquing agent may be incorporated into the powder component as a conventional powder or as nanoparticles.

In one embodiment, the powder composition further comprises an accelerant. Suitable accelerants are discussed above in relation to the powder component of the obturant composition. In some embodiments, the powder composition comprises from 5% to 25% accelerant, especially from 7% to 20% accelerant, more especially from 10% to 15% accelerant, most especially about 10% accelerant.

In another embodiment, the powder composition further comprises an anti-inflammatory agent. In some embodiments, the anti-inflammatory agent is a nonsteroidal anti-inflammatory drug (NSAID) or a corticosteroid, especially a NSAID. Exemplary NSAIDs include ibuprofen, dexibuprofen and lysine ibuprofen, especially ibuprofen and dexibuprofen, more especially dexibuprofen.

In another aspect, the present invention provides a method of making an obturant or liner composition, comprising the step of mixing C₃-C₆ polyol and the powder component discussed above together, wherein the C₃-C₆ polyol comprises from 35% to 65% of the mixture, and the powder component comprises from 35% to 65% of the mixture.

In some embodiments of the method, the mixture comprises from 35% to 45% or 45% to 55% C₃-C₆ polyol, more especially from 35% to 40% or 47% to 53% C₃-C₆ polyol, or from 45% to 50% C₃-C₆ polyol, most especially 37% to 38% or about 50% C₃-C₆ polyol.

Furthermore, in some embodiments of the method, the powder component comprises from 65% to 55% or 47% to 55% of the mixture, more especially from 65% to 60% or 47% to 53% of the mixture, or from 50% to 55% of the mixture, most especially about 62% to 63% or 50% of the mixture.

In another embodiment, the method further comprises mixing an essential oil, such as those discussed above, especially an essential oil and optionally a surfactant with antimicrobial and/or anti-inflammatory properties, with the C₃-C₆ polyol and the powder component. In some embodiments, the essential oil comprises terpinen-4-ol, cinnamaldehyde or carvacrol, or a mixture thereof. In some embodiments, the essential oil comprises from 1.5% to 15% of the mixture, especially about 10% of the mixture. In some embodiments, the essential oil is incorporated into the composition as a powder or as nanoparticles. In other embodiments, the essential oil is incorporated into the liquid component.

In another embodiment, the method further comprises mixing an anti-inflammatory agent with the C₃-C₆ polyol and the powder component. In some embodiments, the anti-inflammatory agent is a nonsteroidal anti-inflammatory drug (NSAID) or a corticosteroid, especially a NSAID. Exemplary NSAIDs include ibuprofen, dexibuprofen and lysine ibuprofen, especially ibuprofen and dexibuprofen, more especially dexibuprofen. The anti-inflammatory agent may be incorporated into the composition as a powder or a nanoparticles.

In one embodiment, this method further comprises the step of mixing calcium hydroxide with a radiopaquing agent and/or an accelerant to prepare the powder component. Suitably, the composition may be mixed by hand or mechanically, for example, with an amalgamator.

The C₃-C₆ polyol, the powder component and optionally the essential oil or the anti-inflammatory agent may be mixed together by any suitable means, for example, in a mechanical agitator, amalgamator or by hand with a spatula.

The setting time of the obturant or accelerated obturant composition may be varied by varying the liquid composition, for example, whether pure C₃-C₆ polyol is included or whether mixtures of polyol and water are used. Varying the ratio of polyol to water varies the setting time. The amount of water in the liquid composition may be from 0% to 60%. The greater the proportion of water in the liquid, the slower the setting reaction. In some embodiments, the liquid component comprises between 10% and 60% water, especially about 20% to 55% water, more especially about 30% to 50% water, for example, 50% water.

In another aspect, the present invention provides the obturant composition or accelerated obturant composition described above for filling or sealing a tooth, a bone cavity, or a bone defect, or for use in the treatment or prevention of a microbial infection in a tooth, a bone cavity, or a bone defect, or for anchoring an artificial joint in place, especially for filling or sealing a tooth.

In a further aspect, the present invention provides a method of filling or sealing a tooth, a bone cavity, or a bone defect, or of treating or preventing a microbial infection in a tooth, a bone cavity, or a bone defect, or for anchoring an artificial joint in place, (especially of filling or sealing a tooth), comprising administering the obturant composition or accelerated obturant composition described above to a subject.

The method especially comprises administering the obturant composition or accelerated obturant composition into the root canal of the subject. The obturant composition may be administered to the root canal of a subject using a lentulo spiral filler or a syringe with a fine cannula. After administration, the obturant composition should occupy all of the space within the root canal. A final restoration may then be placed on top of the obturant composition to restore function to the tooth.

In some embodiments, the obturant or accelerated obturant composition may be used to fill a bone cavity or bone defect or to anchor artificial joints, such as hip, knee, shoulder and elbow joints, in place. The composition of the present invention may replace conventional bone cements currently in use.

As used throughout the specification, the term subject refers to a human or animal in which dental procedures are carried out or those in which bone defects would be repaired or joints replaced. The animal may be a high value animal such as a companion animal or pet or a horse such as a race horse or a captive wild animal such as an animal kept in a zoo. In particular embodiments, the subject is a human.

In another aspect, the present invention provides a use of C₃-C₆ polyol and the powder composition in the manufacture of the obturant composition described above for filling or sealing a tooth, a bone cavity, or a bone defect, or for anchoring an artificial joint in place, or for treating or preventing a microbial infection in a tooth, a bone cavity, or a bone defect; especially for filling or sealing a tooth.

The accelerated obturant composition may also be used in lining cavities in teeth after caries removal, for pulpotomy and pulp capping, for repairing perforations, for achieving a seal in retrograde endodontic procedures or as a bulk restorative for dental temporary or interim restorations.

Advantageously, the obturant composition may be non-staining (colour stable). It may also be used for apexification in immature permanent teeth with open apices or as a bulk filler in permanent teeth that have completed formation. The obturant composition may also be used as a lower cost root canal filler in permanent teeth.

In another aspect, the present invention provides a composition which employs a chemical reaction to generate the calcium hydroxide which then reacts with a C₃-C₆ polyol to form a hard cement material suitable for use as an obturant, pulp capping agent, dentine repair material, or liner for cavity preparations. In the description below this composition is referred to as “the liner composition”.

Advantageously, it has been found that a suitable “feeder” reaction for generating calcium hydroxide is the primary setting reaction of Portland cement as used in industrial concrete, namely a silicate and/or aluminate crystal formation process. The calcium hydroxide generated from this can then to contribute towards the reaction with the C₃-C₆ polyol to give a rapid and biphasic setting reaction. In practical terms, as calcium silicate hydrate and aluminate gels form, the calcium hydroxide generated not only produces an alkaline environment which is unfavourable towards commonly encountered microorganisms in a deep cavity or in the root canal environment, but contributes toward the setting reaction of the bulk material.

Therefore, in this aspect, there is provided a composition comprising a powder composition comprising a silicate and/or an aluminate and a liquid composition comprising C₃-C₆ polyol.

In some embodiments, the silicate is provided by Portland cement, for example, grey or white Portland cement. In particular embodiments, the silicate is calcium silicate or fumed or fine silica. In some embodiments, the aluminate is calcium aluminate or sodium aluminate. Suitable calcium aluminates are provided by the Secar® range of products such as Secar®71, Secar®80, Secar®80F, Secar® Plenium and Secar® Xeniom. For this composition, industrial Portland cement may be used, or an appropriate analogue may be produced using synthetic analytical grade materials rather than raw materials mined from the earth. The required pure materials to produce a chemically pure, ivory-coloured Portland cement free from the presence of heavy metals are as follows: alumina, iron (III) oxide, calcium carbonate, silicon dioxide, and calcium sulphate dihydrate. These are combined, sintered, then ground, to give a powder which is added into the liner composition. The composition may comprise accelerants, radiopaquing agents, essential oils, anti-inflammatory agents as described above for the obturant compositions.

Hydraulic cements consist mainly of silicates and aluminates of lime, and can be classified broadly as natural cements, Portland cements, and high-alumina cements. Calcium aluminates (high alumina cements) are obtained by reacting lime (calcium oxide) and alumina (aluminium oxide) at high temperature to produce a hard material known as calcium aluminate clinker. When this clinker is ground to a powder it is called calcium aluminate cement (CAC).

In contrast, Portland cement is produced by high temperature treatment of a mixture of calcium carbonate (limestone or chalk) and aluminosilicate (clay or shale) and then grinding the reaction product and adding gypsum to produce the final cement powder.

The Portland cement may be included in the powder composition in an amount up to about 33% by weight of the powder composition. Alternatively, calcium aluminate or sodium aluminate may be included in an amount of up to 25% by weight of the powder composition.

The liner composition retains the advantages of a rapid set and a high final surface pH of the “accelerated obturant” composition but with the added advantage that the dentist can now directly manipulate the speed of the setting reaction, across a range from 5 minutes to 12 hours. This is done by choosing pure C₃-C₆ polyol such as glycerol or propylene glycol as the liquid component, or by selecting a liquid component which contains the C₃-C₆ polyol with varying amounts of water, from 0% to 60%. The greater the proportion of water in the liquid, the slower the setting reaction. In some embodiments, the ratio of C₃-C₆ polyol to water is in the range of 60:40 to 40:60, especially 50:50.

For compositions comprising Portland cement, the ratio of powder composition to liquid composition comprising polyol or polyol and water, is in the range of 2.0 to 3.0, especially 2.0 to 2.5. For compositions comprising calcium aluminate, such as the Secar® products, the ratio of powder composition to liquid composition comprising polyol or polyol and water is in the range of 2.5 to 4.0, especially 3.0 to 3.8.

Unlike Biodentine which cannot be hand mixed but must be machine mixed using an amalgamator, the liner composition can be mixed by hand. It is less sensitive to variations in the powder to liquid ratio than either MTA or Biodentine, since the amount of glycerol can be varied by 10% without major problems arising. If the mixture is found to be too dry during hand mixing, glycerol can be added during the mixing phase without adversely affecting the final properties.

The liner composition may comprise an accelerant as described above for obturant compositions. Alternatively, the accelerant may be selected from calcium-sulfo-aluminate (CSA) or calcium aluminate cement (CAC).

The Calcium Sulpho-Aluminate acts as an accelerator, hence its amount can be increased or decreased depending on the desired setting time. Conversely, the content of calcium sulphate hemihydrate can be reduced to extend the working time. Formulations where Portland cement is mixed with Calcium Sulpho Aluminate (CSA) require some water for the initial series of chemical reactions, and will not set if no water is present. A preferred embodiment is described in Example 17 as Mix F and has a water/glycerol ratio of 1:1.

An alternative approach to using CSA is to employ a calcium aluminate cement (CAC). The composition of calcium aluminate products is based on mixtures of aluminium oxide (alumina) and calcium oxide, with lesser amounts of iron oxides and silicon dioxide. The colour of calcium aluminate cements is largely, but not exclusively dictated by the iron oxide content. As a consequence, products in the 40-50% alumina range have darker shades than those with >70% alumina which have a high degree of whiteness. Thus, for a white dental cement powder, a CAC with an alumina concentration of 70% or higher is required.

In some embodiments, especially where high-alumina CAC is used under humid conditions and at temperatures higher than 25° C., a component that reduces the degradation of CAC can be included. Such components include slag and pozzolanic materials such as microsilica and metakaolin. Pozzolans are siliceous, or siliceous and aluminous, materials which will in finely divided form and in the presence of moisture, react chemically with calcium hydroxide to form compounds possessing cement-like properties.

Accelerators/retarders can be added to the above composition to alter the setting time. Calcium aluminate and slag can be used in a ratio between 6:4 and 4:6, for example, in a 60/40 combination or a 50/50 combination. A number of other additives can also be used to reduce the porosity and increase the strength of the set cement. Suitable accelerants for the CAC setting reaction include Portland cement, calcium hydroxide, reground CAC, lithium compounds (e.g. lithium carbonate, nitrate, chloride and hydroxide), and high concentrations of either calcium chloride or magnesium chloride. Suitable retarders include potassium chloride, potassium sulphate, sodium chloride, sodium nitrate and sodium borate, as well as low concentrations of calcium chloride and hydroxycarboxylic acids (citric and tartaric acids and their salts).

As discussed earlier for the obturant composition, for the liner composition, a suitable radiopaquing agent can be selected from suitable non-staining compounds, including zirconium dioxide, barium sulphate, barium zirconate, and calcium tungstate. A suitable range for the radiopaque agent is 15% to 20%. Additional admixtures to the above include fumed silica, synthetic anhydrite, calcium sulphate hemihydrate (an accelerant), calcium sulphate dehydrate (a retardant), lithium compounds (e.g. lithium carbonate, nitrate, chloride and hydroxide as accelerants), and titanium dioxide to lighten the colour of the powder.

In an exemplary embodiment there is provided a liner or obturant composition in which the powder composition comprises 26% Secar®80, 26% slag, 5% silica fume, 10% calcium sulfate (dental stone), 5% sodium aluminate and 28% zirconium dioxide. The liquid composition is a 1:1 mixture of glycerol and water or water. The liquid component to, powder component ratio is 24%. In some embodiments the sodium aluminate may be replaced by CSA.

As described above for the obturant composition, the liner composition can include anti-inflammatory components, such as NSAIDs (e.g. dexibuprofen) and essential oils (e.g. terpinen-4-ol), to extend its therapeutic properties.

In a further aspect, the present invention provides a capsule comprising the powder and liquid components stored separately in different compartments. The materials may, for example, be separated by a membrane which is ruptured when the capsule is activated at the start of the mixing process. The materials in the capsule may then be mixed using an amalgamator.

In a further aspect, the present invention provides a kit comprising the liner composition in a form in which there are a selection of liquids with varying ratios of the C₃-C₆ polyol and water, in order for the dentist to select a desirable final setting time. The powder and liquid portions may be mixed together by any suitable means, for example, in a mechanical agitator or by hand with a spatula.

In another aspect, the present invention provides the liner composition described above for filling or sealing a tooth, a bone cavity, or a bone defect, or for use in the treatment or prevention of a microbial infection in a tooth, a bone cavity, or a bone defect, or for anchoring an artificial joint in place, in a subject, especially for filling or sealing a tooth.

In yet another aspect of the invention there is provided an alkaline resin composition comprising a dental resin and an alkaline calcium compound selected from calcium hydroxide and calcium oxide. In the description below, this composition is referred to as the “dental resin composition”.

In particular embodiments, the dental resin component is Bis-GMA (Bowen's resin) or a resin based on methacrylate or methacrylate ester monomers, such as urethane dimethacrylate (UDMA). The resin may be unfilled or further comprise inert mineral fillers. Furthermore, the resin may further comprise a radiopaquing agent as described above. The alkaline calcium compound is selected from calcium hydroxide or calcium oxide, especially calcium hydroxide. In some embodiments, the calcium hydroxide is nanoparticulate calcium hydroxide.

The alkaline calcium compound is present in an amount of 5% to 50% of the composition, especially 20% to 50% of the composition.

The setting or polymerisation of the dental resin composition may be achieved using a chemical catalyst such as benzoyl peroxide as an initiator and a tertiary amine as an actuator in a two paste system as known in the art. Alternatively, the resin composition may further comprise a photoinitiator such as a camphoroquinone that initiates polymerisation upon exposure to intense blue visible light in the wavelength of 450-500 nm.

Advantageously, inclusion of calcium hydroxide or calcium oxide into dental resin materials and bonding agents achieves the desired release of hydroxyl ions when the set resin comes into contact with water but does not affect the ability of the resin to polymerise and does not affect colour or handling properties such as working time and setting time.

In contrast, use of other alkaline agents such as sodium hydroxide, sodium carbonate, sodium bicarbonate and calcium carbonate, adversely affect viscosity making it unsuitable for application. When sodium hydroxide is used the composition obtained is deliquescent rapidly absorbing water from the atmosphere.

The dental resin compositions of the invention have a pH of at least 9.5, especially at least 10 and the hydroxyl ion release when the set resin comes into contact with water therefore decreases the viability of any bacteria remaining in the site of use in the tooth. In some embodiments, the high pH is achieved while the dental compositions are undergoing their final setting reactions, for several days after placement in the tooth. If post-operative sensitivity caused by ingress of bacteria along the margin of a dental restoration, it will occur predominantly in the first 1 to 10 days after treatment, especially 1 to 7 days.

In yet another aspect of the invention, there is provided an alkaline dental resin composition as described above for use in filling deep carious lesions, pulp capping procedures, pulpotomy, repairing tooth perforations and as a seal in retrograde endodontic procedures.

In yet another aspect of the invention there is provided an endodontic irrigant and/or dental mouthwash composition comprises:
i) calcium hydroxide nanoparticles,
ii) aqueous liquid,
iii) viscosity modifier,
iv) a sweetening agent, and
v) a flavouring agent.

The calcium hydroxide nanoparticles are present in an amount of between 0.1% and 0.4% by weight of the composition, especially about 0.2% by weight.

The pH of the composition is typically above 11.5, especially between 12 and 13, more especially 12 and 12.5.

In some embodiments the aqueous liquid is water. In other embodiments, the aqueous liquid is a mixture of water and ethanol. The mixture may contain up to 25% ethanol.

Viscosity modifiers which are suitable include C₃-C₆ polyols such as glycerol and a low molecular weight polyalkylene glycols such as PEG and PPG, for example PEG or PPG having a molecular weight below 1000, for example between 100 and 500, especially about 200. The viscosity modifier may be present in the composition in an amount of up to 20% by weight. Without wishing to be bound by theory, inclusion of viscosity modifiers increases the amount of calcium hydroxide which may be dissolved into the mixture by over 10 fold, providing benefits in terms of greater hydroxyl ion release. The viscosity modifier also facilitates the incorporation of optional hydrophobic essential oils, such as terpinen-4-ol, cinnamaldehyde, carvacrol, thymol, eucalyptol, menthol, and most especially terpinen-4-ol. This reduces the need for ethanol as a solvent.

In addition to essential oils, other antimicrobial agents are optionally present. Suitable antimicrobial agents for an endodontic irrigant rinse include chlorhexidine acetate and chlorhexidine digluconate (both up to 2%) and triclosan (up to 5%). Suitable antimicrobial agents for an oral mouthrinse for preventing or treating dental diseases include chlorhexidine acetate or chlorhexidine digluconate (both up to 0.2%) and triclosan (up to 0.5%), terpinen-4-ol, cinnamaldehyde, carvacrol, thymol, menthol and eucalyptol (up to 0.2%). Optionally the composition may comprise anti-inflammatory agents such as NSAIDS or corticosteroids as described above.

The flavouring agent may be any flavouring agent that is generally considered safe (GRAS) for example, peppermint, spearmint, almond, orange, strawberry, raspberry, cherry, banana, and the like. The flavouring agent may be added in an amount of between 0.5% and 1% by weight of the composition. The most preferred flavour combination is almond with either peppermint or spearmint, at a ratio of 3:1. It has been found that this particular flavour combination nullifies any brackish taste from the alkaline rinse and obviates the potential for soft tissue irritation, particularly in patients who have impaired salivary production who experience irritant reactions from most commercial mouthwashes.

The sweetening agent is preferably an artificial sweetening or intense agent such as aspartame, saccharine or stevia (steviol glycoside). The sweetening agent may be added in an amount of 0.1% to 1%, especially 0.1% to 0.5% by weight of the composition.

In some embodiments, the composition comprises a light absorbing dye in an amount of up to 1.0 mg/mL. Suitable dyes include tolonium chloride, methylene blue, phenothiazine dyes and rhodamine dyes.

The endodontic disinfectant or irrigant solution may be particularly useful in disinfecting the root canal during endodontic treatment.

In another aspect of the invention there is provided a method of disinfecting the root canal of teeth comprising rinsing the root canal with an aqueous endodontic irrigant composition comprising calcium hydroxide nanoparticles.

The method may be used to disinfect the root canal system during endodontic treatment. The nanoparticles may be rinsed passively through the root canal system or may be assisted by agitation. Suitable agitation may be achieved by ultrasound applied to a tooth with a piezoelectric element or magnetostrictive handpiece or by use of a pulsed laser, such as a diode laser or solid state laser operating in the near infrared or middle infrared region.

Without wishing to be bound by theory, it is thought that cavitation forces nanoparticles into dentine tubules as well as enhancing microbial contact throughout the root canal system and thereby improves antimicrobial activity.

In some embodiments, the endodontic irrigant composition comprises other components such as viscosity modifiers, antimicrobial agents, flavouring agents, sweetening agents and light absorbing dyes as described above.

In another aspect of the invention there is provided a method of disinfecting the entire oral cavity to lower the level of acid tolerant microorganisms, which flourish overnight and at periods during the day when the resting output of saliva falls. Levels of acid-tolerant bacteria and fungi are high in patients whose salivary outputs are reduced because of medical treatments (such as head and neck radiotherapy), medicines, or salivary gland diseases. Frequent use of conventional mouthrinses is not possible in such patients because of the problems of staining and irritation. Rinsing the mouth with an aqueous mouthwash composition comprising calcium hydroxide nanoparticles, a sweetener, a viscosity modifier and a preferred flavour combination of almond/peppermint at a ratio of 3:1 has been found to be well tolerated by dental patients suffering from dental caries, dental erosion, dental root surface sensitivity, and dry mouth, without attendant staining or irritant reactions.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Unless stated otherwise, the percentages appearing within this specification and the claims which follow represent percentages by weight.

The invention will now be described with reference to the following Examples which illustrate some aspects of the present invention. However, it is to be understood that the particularity of the following Examples is not to supersede the generality of the preceding description of the invention.

### EXAMPLES

### Example 1

### Endodontic Medicament Composition

PEG 400 liquid (76 grams) and PEG 3350 (4 grams) were added to a stainless steel vessel, and the resultant mixture was heated at 60° C. with constant stirring until the PEG 3350 had melted and dissolved evenly into the mixture, over a period of approximately 2 to 3 minutes.

The mixture was then removed from heat, and then the following powders were added in sequence with gentle stirring: (i) calcium hydroxide powder (65 grams), (ii) zirconium dioxide powder (25 grams), and (iii) titanium dioxide powder (3.5 grams). The final pH of the set product was measured 5 minutes after inserting a pH electrode, achieving a pH of 13.5.

The cooling mixture was then mechanically stirred for a period of 5 minutes, and then the mixture was dispensed into tubes or syringes.

### Example 2

### Endodontic Medicament Comprising Terpinen-4-ol Anti-Inflammatory Agent

The same procedure as Example 1 was followed with the exception that when the mixture was removed from the heat, the following components were added with gentle stirring: i) calcium hydroxide powder (72 g), ii) zirconium dioxide powder (33 g), iii) titanium dioxide powder (2.5 g) and iv) terpinen-4-ol (22 g).

### Example 3

### Endodontic Medicament Comprising Dexibuprofen Anti-Inflammatory Agent

The same procedure as Example 2 was followed with the exception that the terpinen-4-ol was replaced with dexibuprofen (22 g).

### Example 4

### Endodontic Obturant Composition

A powder was prepared by mixing together calcium hydroxide, calcium sulphate hemihydrate (dental plaster), and barium sulphate in a stainless steel vessel. For every 100 g of powder, there is 40 g of calcium hydroxide, 10 g of dental plaster and 50 g of barium sulphate.

This powder was gradually added to the same amount by weight of glycerol (i.e. for 0.5 g of powder, 0.5 g of glycerol was used) on a glass mixing slab at room temperature. After all of the powder had been incorporated, the composition was ready for use.

### Example 5

### Endodontic Obturant Composition with Anti-Inflammatory Agent

A powder was prepared by mixing together calcium hydroxide, calcium sulphate hemihydrate (dental plaster), barium sulphate and dexibuprofen in a stainless steel vessel. For every 100 g of powder, there is 40 g of calcium hydroxide, 8 g of dental plaster, 10 g of dexibuprofen and 40 g of barium sulphate.

This powder was gradually added to the same amount by weight of propylene glycol (i.e., for 0.5 g of powder, 0.5 g of propylene glycol was used) on a glass mixing slab at room temperature.

After all of the powder had been incorporated, the mixture was stirred with a spatula to form an even paste. The composition was then ready for use.

### Example 6

### Preparation of Calcium Hydroxide Nanoparticles

Calcium hydroxide nanoparticles were prepared based on the method of Danielea and Taglieri (Journal of Cultural Heritage (June 2011), doi:10.1016/j.culher.2011.05.007). Briefly, 4.41 g (0.03 moles of calcium chloride dihydrate was dissolved into 100 mL of deionized water, together with 1% w/w Tween 80 based on the weight of calcium chloride solution. A solution of sodium hydroxide was prepared by dissolving 2.4 g into 100 mL of water. The calcium chloride solution and sodium hydroxide solution were warmed separately to a constant temperature of 50° C. then mixed together in bulk to give a milky solution of about 200 mL. The calcium hydroxide precipitates and the particles settle at the bottom of the container.

After 60 minutes, the supernatant was removed by decantation and the calcium hydroxide solid nanoparticles having an average size range of between 50 and 500 nni were washed with distilled water (100 mL) by agitation for 30 to 120 minutes. The wash water was then decanted. The pH of the nanoparticles of calcium hydroxide in a solution of 500 mL distilled water was greater than 12.

### Example 7

### Endodontic Medicament Composition

The steps of Example 1 were repeated but the calcium hydroxide powder was replaced with 65 g of thick suspension of calcium hydroxide nanoparticles prepared in Example 6. The final pH was 13.5.

### Example 8

### Endodontic Disinfectant and Irrigant

An endodontic disinfectant solution for use as a rinse solution was prepared by suspending calcium hydroxide nanoparticles in aqueous solution to give a final concentration of calcium hydroxide of 0.2%. The final pH of the composition was 12.5.

### Example 9

### Mouthwash

The supernatant obtained from the production of calcium hydroxide nanoparticles in Example 4 was diluted five fold and 0.5% w/w peppermint flavouring and 0.3% w/w stevia were added. The solution was mixed. The final pH of the composition was 12.2.

### Example 10

### Mouthwash

The supernatant obtained from the production of calcium hydroxide nanoparticles in Example 4 was diluted five fold and from Example 7 and 0.1% w/w peppermint flavouring, 1% w/w sodium saccharine and 0.5% almond flavouring was added. The solution was mixed. The final pH of the composition was 12.0.

### Example 11

### Mouthwash with Enhanced Antimicrobial Properties

The supernatant obtained from the production of calcium nanoparticles in Example 6 was diluted 5-fold and 4% w/w of stevia was added. A 10 times concentrated stock solution of PEG200 was prepared containing 2% terpinen-4-ol. This stock solution was diluted ten-fold with the calcium hydroxide nanoparticle solution to give a mouthwash composition with a pH of 11.6.

### Example 12

### Mouthwash with Enhanced Antimicrobial Properties

The supernatant obtained from the production of calcium hydroxide nanoparticles in Example 6 was diluted 5-fold and 6% w/w stevia was added. A ten times concentrated stock solution of PEG200 was prepared with a final concentration of 2% terpinen-4-ol, 2% carvacrol acid 2% cinnamaldehyde. This stock solution was then diluted 10 fold with the calcium hydroxide nanoparticle solution to give a mouthwash composition with a pH of 11.6.

### Example 13

### Dental Resin

100 mg of light cured flowable resin (Southern Dental Industries, Melbourne, Australia) was enriched with either calcium oxide or calcium hydroxide to a final concentration of 5% by weight. The composition was mixed thoroughly for 20 seconds. The composition was then placed in a mould. The resin was polymerised by exposing the mould to blue light at 470 nm for 40 seconds. The polymerised resin material set to the same hardness as unmodified resin. Samples of each polymerised resin having similar mass and surface area (150 mg) were placed in test tubes and covered with 1 mL of water. The pH of the water was tested at 60 second, 30 minutes, 3 days and 2 weeks. The results are shown in Table 1.

TABLE 1
pH achieved of resins

| Time | Unmodified resin | 5% calcium oxide | 5% calcium hydroxide |
|---|---|---|---|
| 60 seconds | 7.76 | 10.00 | 10.77 |
| 30 minutes | 7.77 | 10.30 | 11.30 |
| 3 days | 7.76 | 10.00 | 11.54 |
| 2 weeks | 7.74 | 9.19 | 10.81 |

Similar results were obtained using Bis-GMA bonding agents with calcium hydroxide.

### Example 14

### Alkaline Lining Cement Composition

A powder composition was prepared containing 17% w/w calcium hydroxide, 17% w/w calcium sulphate hemihydrate, 23% w/w barium sulphate, 23% w/w sodium aluminate, 9% calcium chloride and 11% fumed silica. The powder composition was mixed with glycerol at a powder to liquid ratio of 1.67 w/w to give a rapidly setting material which has a working time of 2 minutes. This composition set hard within 2 hours.

### Example 15

### Alkaline Lining Cement Composition

5 g fine silica is combined with 1.5 g of calcium chloride, 4 g of sodium aluminate, 3 g of calcium sulphate hemihydrate and 4 g zirconium dioxide to give 17.5 g powder composition. The powder composition is mixed with 11 g glycerol for 30 seconds to give a creamy mix. At the start of mixing, the mix will appear to be too dry, but after. 10 to 15 seconds of mixing, a stiff creamy mix will be obtained. The working time of this mix is 1.5 minutes, and the setting time is 5.0 minutes. These setting times are sufficient short to allow the set liner to be overlaid with a suitable permanent restorative material, such as a resin composite or a dental ceramic.

### Example 16

### Slow and Fast Setting Dental Cements

Examples of slow and fast setting alkaline dental cements are shown below, each of which uses different levels of accelerants and feeder reactions (amounts shown are in grams). Mixes A, B and E lack added calcium hydroxide in the powder, while Mixes A-D use Portland cement to generate calcium hydroxide.

| | Mix A | Mix B | |
|---|---|---|---|
| Calcium | 0 | 0 | |
| hydroxide | | | |
| White Portland 8 | 8 | | |
| cement | | | |
| Zirconium | 3 | 3 | |
| dioxide | | | |
| Sodium | 0 | 2 | |
| aluminate | | | |
| Distilled water | 1.5 | 1.7 | |
| Glycerol | 1.5 | 1.7 | |
| Working time | 10 | 4 minutes | |
| minutes | | | |
| | Mix C | Mix D | Mix E |
| White Portland | 20 | 20 | 10 |
| cement | | | |
| Calcium | 5 | 10 | 0 |
| hydroxide | | | |
| Calcium oxide | 0 | 0 | 5 |
| Calcium | 7 | 7 | 3 |
| sulphate | | | |
| hemihydrate | | | |
| Silica | 2 | 2 | 2 |
| Barium | 10 | 10 | 5 |
| sulphate | | | |
| Glycerol | 23.8 | 29 | 15.5 |
| Setting time to | 3 hours | 2 hours | 6 hours |
| final hardness | | | |

Unlike MTA or Biodentine, the essential liquid ingredient of the liner composition is glycerol, rather than water. This gives a smoother mix for the dentist to handle, and prevents the mixed material drying out during its application onto or into the tooth. The liner composition handles better as it has a creamy rather than a sandy consistency. Because the liner composition has a better consistency of flow compared to the water based MTA and Biodentine, the mix can be placed into difficult to reach regions and can be packed into place if desired.

### Example 17

### Use of CSA as an Accelerant in a Liner Composition

The following are examples of formulations of the liner composition where Portland cement is mixed with Calcium Sulpho Aluminate (CSA). The addition of CSA to Portland cement gives rapid setting and high early strength development, features which are essential for use of the material as a liner or as a temporary filling material.

| | Mix F | Mix G |
|---|---|---|
| White Portland cement | 14 | 14 |
| Calcium Sulpho-Aluminate | 5 | 5 |
| Calcium sulphate | 5 | 5 |
| hemihydrate | | |
| Silica | 2 | 2 |
| Zirconium dioxide | 7 | 7 |
| Distilled water | 7 | 15 |
| Glycerol | 7 | 0 |
| Setting time | 5 minutes | 2 minutes |

### Example 18

### Calcium Aluminate-based Dental Liner or Obturant

A powder composition comprising:

| | |
|---|---|
| Secar 71 or 80 (containing 71% or 80% alumina respectively) | 5 g |
| Slag (ground granulated blast furnace slag) | 5 g |
| Silica Fume | 1 g |
| Calcium Sulphate Hemihydrate | 2 g |
| Zirconium dioxide | 5 g |
| Sodium aluminate | 1 g |

was mixed and then 6 mL of a 1:1 mixture of glycerol and water was added with mixing. The use of glycerol and water allows the CAC setting reaction to occur and tempers the speed of setting.

### Example 19

### Portland Cement based Dental Liner or Obturant

A powder composition comprising:

| | |
|---|---|
| Portland cement | 14 g |
| Calcium Sulfo-Aluminate (CSA) | 7 g |
| Calcium Sulphate (dental stone) | 5 g |
| Silica, | 2 g |
| Zirconium dioxide | 7 g |

was mixed and then a mixture of 8 g glycerol and 7 g water was added with mixing.

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

1. A composition comprising a polyalkylene glycol solvent and calcium hydroxide, wherein the pH of the composition is at least 13.0 and wherein the polyalkylene glycol solvent has a molecular weight of from 200 to 600 or a mixture thereof and is present in an amount of from 30% to 60% of the composition and wherein the calcium hydroxide is in an amount of from 25% to 55% of the composition.

2. The composition according to claim 1, wherein the pH of the composition is at least 13.5.

3. A composition according to claim 1 wherein the polyalkylene glycol solvent is polyethylene glycol or polypropylene glycol.

4. (canceled)

5. The composition according to claim 1, wherein the polyalkylene glycol solvent is polyethylene glycol 400 or polypropylene glycol 400.

6. (canceled)

7. (canceled)

8. The composition according to claim 1, further comprising a viscosity modifier, wherein the viscosity modifier is a polyalkylene glycol with a molecular weight of from 2800 to 4000 or a mixture thereof.

9. (canceled)

10. The composition according to claim 8, wherein the viscosity modifier is polyethylene glycol 3350 or polypropylene glycol 3350.

11. The composition according to claim 1 further comprising a radiopaquing agent.

12. The composition according to claim 11 wherein the radiopaquing agent is zirconium dioxide, barium sulphate or barium zirconate.

13. The composition according to claim 1 further comprising at least one anti-inflammatory agent.

14. The composition according to claim 13 wherein the anti-inflammatory agent is selected from essential oils and NSAIDS.

15. A method of making the composition defined in claim 1 comprising adding calcium hydroxide to a polyalkylene glycol solvent, and mixing the combination.

16. A method of treating or preventing a microbial infection in a tooth, painful extirpation, “hot pulp” syndrome or acute pulpitis, or a combination thereof, comprising administering the composition defined in claim 1 to a subject.

17-60. (canceled)
